(19) United States
(12) Patent Application Publication
(10) Pub. No.: US 2010/0310519 A1
(43) Pub. Date: Dec. 9, 2010

(54) Incorporation of Active Molecules into Microalga

(76) Inventors: Henri Oreal, La Seyne sur Mer (FR); Jean-Pierre Brune, La Valette (FR)

Correspondence Address: MCANDREWS HELD & MALLOY, LTD, 500 WEST MADISON STREET, SUITE 3400, CHICAGO, IL, 60661, US

(21) Appl. No.: 12/445,612

(22) PCT Filed: Oct. 11, 2007
(86) PCT No.: PCT/FR07/01660

§ 371 Date: Aug. 18, 2010

(30) Foreign Application Priority Data
Oct. 19, 2006 (FR) 0609145

(51) Int. Cl. A61K 35/74; C12N 5/02; C12N 13/00; A61P 35/00

(52) U.S. Cl. 424/93.4; 435/373; 435/173.1

(57) ABSTRACT

Process of incorporating active and aggressive molecules into a microalga, with targeted recognition of cancer cells. The invention relates to the incorporation of active molecules into a microalga having first developed a plasmid, and/or a gene with resistance to the specific molecules. The process of the invention is particularly intended for human and veterinary medicine.

The present invention describes a method allowing the addition of active molecules to a culture medium for microalgae, in order to propose vectors of molecules allowing a considerable reduction in the side effects of chemotherapy whilst maintaining the latter at its highest bioavailability level.

Under normal circumstances, cytotoxic molecules such as antimetabolites which act by preventing the synthesis of different molecules involved in the DNA system and alkylating agents which are molecules containing alkyl groups as well as extracts from microorganisms such as daunorubicin lead to the apoptosis of cancer cells; unfortunately not all these agents are specific to the cancer cell and so-called normal cells are considerably affected, in particular the blood cell line.

These chosen vectors are cyanobacteria.

The medicaments and the molecules are not combined but added to microalgae.

The molecules are essentially anticancer agents and among the chosen anticancer agents there can mentioned taxotere, the kinase inhibitors, flavopiridol, roscovitine, endoxan, doxorubicin or ricin A etc.

These active molecules, by their composition, induce a dysfunction in the cell diffusion of the vector, they cannot therefore be used as they are.

Among the chosen microalgae we shall essentially use spirulina (arthrospira platensis) which has been authorized for human and animal consumption by the Council for Public Health in France.

This cyanobacterium by itself has known effects on the human organism. It is very high in protein (65%), contains vitamins, minerals, polysaccharides, peptides and pigments.

Moreover they have important functions in particular being able to naturally bind molecules by ionization or chemical processes, but as regards anticancer molecules they cannot induce or bind such aggressive molecules, without having expressed an optional selection of a mutant form of cyanobacterium which represents a resistance to these anticancer molecules.

The subject of the invention is the development of an optional selection of a mutant form in the cyanobacteria and in particular spirulina (athrospira platensis) characterized in that it comprises a step of culturing the microalgae in an alkaline culture medium essentially containing a homogenate of neoplastic cells which have been subjected to thermal shock.

The culture of the microalga takes place monoclonally until a useful biomass is obtained, the neoplastic cells originating from humans having been subjected to all the existing viral ranges (HTV3 hepatitis B, hepatitis C, herpes, megalo-virus etc.).

A more particular subject of the invention is a method for physiological induction and addition and internalization of anticancer molecules characterized in that it comprises several steps.

In particular, after the culture of microalgae in the presence of neoplastic cells said cyanobacteria are re-cultured in an appropriate medium of sodium chloride and anticancer molecules in a dilution of 1 to 5-thousandths. This method of addition of specific anticancer molecules is preceded by decreasing dilutions in the appropriate culture medium containing a solution, therefore the composition in gr/litre is as follows:

| | |
|---|---|
| NaOHCO₃ | 15.6 |
| K₂HPO₄ | 0.6 |
| NaNO₃ | 2.1 |
| K₂SO₄ | 0.9 |
| NaCl | 1.6 |
| MgSO₄ | 8 |
| HO₂ | 0.2 |
| CaCl₂ | 0.04 |
| FeSO₄ | 7 |
| EDTA | 0.08 |
| ZnSO₄ | 7 |
| CuSO₄ | 5 |
| NiSO₄ | 0.047 |

sufficient quantity of physiological water

The active molecules are diluted in this solution, for example:

1/5000, 1/2500, 1/1250.1/625, 1/300, 1/100, 1/50, 1/10, 1/5.

The cyanobacterium in decreasing contact with the anticancer molecules will give rise to an optional selection of a mutant form which can easily be verified since it will accept these said molecules or a resistance gene which can be verified by comparison or by simple karyotyping. The cyanobacterium will then have a useful load which can be used for the treatment of diseases (human or veterinary medicine). The doses can be adjusted as a function of the weight of the patient and seriousness of the disease.

The present invention essentially follows from the development of an optional selection of a specific mutant form and/or of an optional gene of resistant to a given antimitotic; since there cannot be any incorporation of so-called aggressive molecules without having to develop an optional selection of a mutant form beforehand.

The microalga protein, having recognized the neoplastic protein several times, will specifically recognize the cancer cells which it will have as a target. This will have an antibody receptor effect. These proteins loaded with antimitotic will cause the cancer cells to implode (by acting on the different cell division phases) without (or very slightly) attacking the other cells while avoiding the plasmatic peaks and side effects due to chemotherapy.

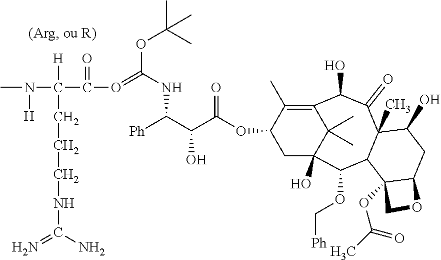

Example of incorporation of an active cytotoxic molecule such as taxotere into an amino acid.

In order to facilitate this incorporation of the antimitotic molecule into the amino acid, the culture medium will then be subjected to the action of an electromagnetic field pulsed at high frequency (1 GHz) of a few kV.cm⁻¹. It will follow from this that certain portions of the protein molecule will resonate and turn freely (so-called δ relaxation). The levels of activation energy of the chemical bonds then being compatible, the bindings of the macromolecules will be greatly facilitated.

1. A method for preparing a culture of cyanobacteria, wherein said method comprises (i) a step of culturing cyanobacteria in an appropriate culture medium containing mineral salts and a homogenate of cancer cells allowing a sufficient biomass, and (ii) a step of addition of the anticancer molecules to the culture medium wherein said cyanobacteria exhibit resistance to the anticancer molecules and become loaded with said anticancer molecules.

2. The method according to claim 1, wherein the culture medium is an alkaline medium.

3. The method according to claim 1, wherein said cancer cells have been subjected to a thermal shock.

4. The method according to claim 1, wherein the addition of the anticancer molecules is achieved by a successive dilution of the anticancer molecules in said culture medium; in order to express an optional selection of a mutant form of cyanobacteria which represents a resistance to these anticancer molecules.

5. The method according to claim 1, wherein said method further comprises (iii) a step of exposing the culture medium to an electromagnetic field pulsed at high frequency.

6. A pharmaceutical composition for human or veterinary use comprising a culture of cyanobacteria obtained by the method according to any one of claims 1 to 5.

7. The composition of claim 6 wherein said cyanobacteria is Arthrospira platenis.

8. The method according to any one of claims 1 to 5 wherein said cyanobacteria is Arthrospira platenis.
